SEQUENCE LISTING (11 sequences)

SEQ ID NO: 1 - DNA, 20 bp, Artificial
  Reporter Oligonucleotide (RO-TAMRA). The 5′- TAMRA-labeled oligonucleotide is complementary to the 5′ tail sequence of the capture oligonucleotide.
aaaatccacccaccccaccc

SEQ ID NO: 2 - DNA, 20 bp, artificial
  Reporter Complement (RC). This oligonucleotide is complementary to the reporter oligonucleotide.
gggtggggtgggtggatttt

SEQ ID NO: 3 - DNA, 79 bp, artificial
  Capture Oligonucleotide (CO) is a 79-mer oligonucleotide has a short nucleotide sequence complementary to a sequence in the murine B7.2 mRNA.
gggtggggtgggtggattttcccaaacttacggatcgtgggtgcttccgtaagtttgggcccctcctcctccctcctcc

SEQ ID NO: 4 - DNA, 79 bp, artificial
  Control Capture Oligonucleotide (CCO). This oligonucleotide has the same sequence as the capture oligonucleotide except that three thymines replace three guanines at positions 23 to 25 (from the 5′ terminus).
gggtggggtgggtggattttaaaaaacttacggatcgtgggtgcttccgtaagttttttcccctcctcctccctcctcc

SEQ ID NO: 5 - DNA, 24 bp, artificial
  24mer Target Sequence (24mer).  This oligonucleotide represents a target that is complementary to 24 nucleotides in the target recognition sequence in the CO and CCO.
cccaaacttacggaagcacccacg

SEQ ID NO: 6 - DNA, 67 bp, artificial
  B7-67mer Target Sequence (B7-67mer). This oligonucleotide represents a segment of the murine B7.2 mRNA sequence. Its sequence is complementary to the 22 nucleotides in the mRNA recognition sequence.
ccagaacttacggaagcacccacgatggaccccagatgcaccatgggcttggcaatccttatctttg

SEQ ID NO: 7 - DNA, 20 bp, artificial
  Address Oligonucleotide with Disufide (AO/SS). This oligonucleotide has a disulfide group at the 5′ end that enables its attachment to the substrate.
ggaggagggaggaggagggg

SEQ ID NO: 8 - DNA, 70 bp, artificial
  Capture oligonucleotide (CO) sequence used in Example 6.
gggtgggtgggtggttattttcccttacatcgtgggtgcttccgtaagggtgggagggagggagggagag

SEQ ID NO: 9 - DNA, 67 bp, artificial; identical to SEQ ID NO: 6
  B7-67mer sequence is identical to SEQ ID NO6, which represents a segment of the murine B7.2  mRNA sequence.

SEQ ID NO: 10 - DNA, 15 bp, artificial
  T3 sequence complementary to the CO loop region
ggaagcacccacgat

SEQ ID NO: 11 - DNA, 15 bp, artificial
  SM sequence differs from the T3 sequence in only one base at position 6.
ggaagaacccacgat

(19) United States
(12) Patent Application Publication
(10) Pub. No.: US 2008/0293580 A1
(43) Pub. Date: Nov. 27, 2008

(54) Methods for Detecting and Measuring Specific Nucleic Acid Sequences

(76) Inventors: Suk-Wah Tam-Chang, Reno, NV (US); Kenneth W. Hunter, Reno, NV (US); Nelson G. Publicover, Reno, NV (US)

Correspondence Address: NIXON PEABODY, LLP, 401 9TH STREET, NW, SUITE 900, WASHINGTON, DC, 20004-2128, US

(21) Appl. No.: 10/578,248

(22) PCT Filed: Nov. 8, 2004
(86) PCT No.: PCT/US04/37041

§ 371 Date: Feb. 27, 2007

Related U.S. Application Data
(60) Provisional application No. 60/517,399, filed on Nov. 6, 2003.

(51) Int. Cl. C40B 30/04; C40B 40/08; C07H 21/00

(52) U.S. Cl. 506/9; 506/17; 536/22.1

(57) ABSTRACT

The invention provides novel oligonucleotides and methods of using the same for detection or measurement of specific nucleic acid molecules. The invention also features nucleic acid arrays comprising the oligonucleotides of the invention. An oligonucleotide of the invention comprises (1) a reporter-binding sequence capable of hybridizing to a fluorrophore-labeled reporter sequence and (2) a hairpin-forming sequence capable of forming a stem-loop. Formation of the stem-loop modifies (e.g., quenching) the fluorescence signals of the reporter sequence when the reporter sequence is hybridized to the oligonucleotide. This can be achieved, for example, by bringing one or more guanine based in the oligonucleotide into close proximity to the fluorophore(s) of the reporter sequence by virtue of the formation of the stem-loop. Disruption of the stem-loop, such as by hybridization of a target sequence to at least part of the hairpin-forming sequence, produces a detectable change in the fluorescence signals.

### CROSS-REFERENCE TO RELATED APPLICATIONS

The present application claims the benefit and incorporates by reference the entire disclosure of U.S. Provisional Application No. 60/517,399, filed Nov. 6, 2003.

### FIELD OF THE INVENTION

The present invention relates generally to the detection and measurement of nucleic acids. More particularly, it relates to a novel method of detecting and measuring specific sequences of nucleic acids from biological materials.

### BACKGROUND OF THE INVENTION

The detection and measurement of specific nucleic acid sequences have become an important tool for basic genetic research, medical and veterinary diagnosis/prognosis, and forensic science. A number of techniques have been developed to detect and measure nucleic acid sequences, however, with the recent publication of the sequence of the human genome as well as the sequences of a variety of non-human genomes ranging from mice to bacteria, considerable excitement has been engendered by the so-called gene microarray technology. This relatively new technology offers the potential of simultaneously detecting and measuring thousands of nucleic acid sequences. Indeed, the human genome has approximately 35,000 genes, and gene microarray technology has the potential of enabling the detection and measurement all human genes simultaneously. The notion behind this technology is to utilize the inherent ability of single stranded nucleic acids (both deoxyribonucleic acid or DNA and ribonucleic acid or RNA) to hybridize to a complementary single-strand oligonucleotide sequence through Watson-Crick base-pairing in order to detect the presence and amount of specific nucleic acid sequences in biological samples.

One of the most exciting areas to which gene microarray technology is being applied is functional genomics. While knowledge of the sum total of the genes in a genome is extremely important, it is perhaps more important to know which of these genes are functioning at a particular time in a particular cell or tissue. For genes represented in the DNA to function they must first be transcribed into messenger RNA (mRNA), and the mRNA must then be translated into protein. By measuring specific mRNA sequences one can determine which of the genes represented in the genome are functioning at a particular moment in time. Many disease states in humans and animals are characterized by a change in gene function in certain cells or tissues, and detection of the pattern of gene expression is useful, therefore, for both diagnosis and prognosis.

In a common embodiment of the gene microarray technology, RNA is removed from cells or tissues by an extraction procedure, and then after purification of the mRNA it is subjected to reverse transcription, an enzymatic process whereby the mRNA is converted into a complementary DNA (cDNA). During this reverse transcription process either fluorophore-labeled nucleotides, or nucleotides with chemical side-groups that allow fluorophores to be attached, are added. After the cDNA is fluorophore-labeled, it is added to a detection system usually involving a complementary oligonucleotide attached to a solid substrate. Under conditions suitable for hybridization, the fluorophore-labeled cDNA is “captured” to the solid surface by complementary base pairing, and then following a wash step to remove the non hybridized cDNA, the hybridized cDNA is measured by one of several standard fluorimetric techniques. The end result of this procedure is the detection, and in some cases, quantification of the expression of specific genes by the measurement of their mRNAs.

Notwithstanding the attributes of modern gene microarray technology, it has many problems that need new solutions. For instance, when used for gene expression analysis, the mRNA to be measured must be purified from the cells or tissue and then converted enzymatically into cDNA in order to add the fluorophore. This is a time consuming technique that requires a sophisticated laboratory with expensive equipment and reagents. Moreover, because the method involves a washing step to remove the unhybridized cDNA, it cannot be performed in a single step.

### BRIEF SUMMARY OF THE INVENTION

The present invention provides improved methods for detecting and measuring specific nucleic acids. In one aspect, the present invention provides nucleic acid detection methods that are rapid and cost-effective. In another aspect, the present invention provides in vitro, in vivo, or in situ diagnostic methods for quantitatively measuring multiple, specific nucleic acid sequences from biological samples.

According to some embodiments, the nucleic acid detection and measurement methods of the present invention do not require RNA purification, production of cDNA by reverse transcription, or chemical labeling of the nascent cDNA strand. Moreover, the methods can be reversible and do not require a wash step; thus they are suitable for real-time in vivo or in situ gene expression analysis.

Like the majority of nucleic acid detection methods using gene microarrays, many embodiments of the invention use a single-stranded DNA capture oligonucleotide with a sequence complementary to that of the nucleic acid to be measured. This capture oligonucleotide can also be other detectable nucleic acid molecules, such as RNA, 2′-O-methyl oligoribonucleotide, or have a chemically modified backbone such as a backbone based on peptide linkages or a backbone with phosphorothiolates instead of phosphate groups. This capture oligonucleotide can be used either in solution or attached to a substrate support. Various methods are known in the art for attachment of an oligonucleotide to a substrate support. The attachment can be covalent or non-covalent. For instance, a functional chemical group can be incorporated into the oligonucleotide. The functional chemical group can form a covalent bond to another functional chemical group on the support. Examples include, but are not limited to, functional groups that can be incorporated into the stem-loop strand using phosphoramidite precursors in standard solid phase synthesis. Some specific examples of these function groups are: thiol or disulfide groups for self-assembly onto a metal (e.g. gold, silver, copper) surface or for reaction with a substrate surface that is derivatized with thiol or disulfide reactive groups (e.g. acrylamide, epoxide, thiol); an amino group for reaction with a substrate surface that is derviatized with amino reactive groups (e.g. carboxylic acids, succinimides, anhydrides); or an acrylamide group for reaction with a substrate surface that is derviatized with acrylamide reactive groups (e.g. thiols). The following references are provided as examples of methods of attaching oligonucleotides to substrate supports: Fodor et al., U.S. Pat. No. 5,744,305; Beier and Hoheisel (1999) Nucleic Acids Research 27:1970-1977; Niemeyer and Blohm (1999) Angew. Chem. Int. Ed. 38:2865-2869; Rogers, Y-H. et al. (1999) Anal. Biochem. 266: 23-30; Schena, M. “DNA Microarrays. A Practical Approach”, Oxford University Press, New York, N.Y., (1999); Schena, M. “Microarray Biochip Technology”, Eaton Publishers., Natrick, Mass., (2000); Zammatteo, N. et al. (2000) Anal. Biochem. 280:143-150; Pirrung, M. C. (2002) Angew. Chem. Int. Ed. 41: 1276-1289; Charles, P. T. et al. (2003) Langmuir 19:1586-1591. Schena, M. “Microarray Analysis”, Wiley-Liss, Hoboken, N.J., (2003), all of which are incorporated herein by reference in their entireties.

In one embodiment of the invention, the capture oligonucleotide is not directly attached to a surface, but rather has a 3′ sequence complementary to that of an address oligonucleotide that is chemically attached to the surface. This attachment means avoids the expense of having the longer capture oligonucleotide chemically modified at its 3′ end to enable chemical attachment directly to the surface. Moreover, this facilitates the easy self-assembly of the capture oligonucleotides on the substrate surface. Another feature of the single-stranded capture oligonucleotides of this embodiment is that they have base sequences that cause them to form hairpins or stem-loops at room temperature. However, when these capture oligonucleotides hybridize with a complementary nucleic acid strand, the single-stranded capture oligonucleotides can no longer form the hairpin or stem-loop secondary structures but remain in the linear configuration. Another feature of the capture oligonucleotides of this embodiment is that they include 5′ tail segments with a common sequence that enables the hybridization of a fluorophore-labeled reporter oligonucleotide sequence. When the hairpins are in the open configuration, excitation of the fluorophores attached to the hybridized reporter oligonucleotides results in a characteristic emission of photons (fluorescence). However, when the capture oligonucleotides are in the closed hairpin or stem-loop configuration, the fluorophores on the reporter oligonucleotides are brought into close proximity to guanosine bases strategically placed 3′ to the point where the hybridized bases form the hairpin or stem-loop (hairpin-forming sequences). Upon excitation of the fluorophores under these conditions, the fluorescence emissions are quenched. This quenching preferably is reversible.

Other quenching mechanisms can also be used in the present invention. For instance, photoinduced electron transfer (or photoinduced charge transfer) may occur between luminescence compounds (fluorescence, phosphorescence, and electroluminescence) or luminescent nanoparticles and naturally occurring nucleotides (e.g., guanosine nucleotides), synthetic nucleotide analogs, other synthetic quenchers (including quenchers that intercalate into DNA or RNA duplexes, or quenchers that can be incorporated into an oligonucleotide from a precursor such as a phosphoramidite monomer by solid-phase oligonucleotide synthesis), or metals (e.g., bulk or nanoparticles) as quenchers. Some examples are illustrated in (but not exclusive to) the references below: Schena et al., (1995) Science 270:467-470; Claus, et al. (1996) J. Phys. Chem. 100:5541-5553; Lewis and Wu, (2001) J. Photochem, and Photobiol. C: Photochem. Rev. 2: 1-16; Lewis, et al. (2001) Acc. Chem. Res. 34:159-170; Prasanna de Silva et al. (2001) Trends in Biotechnol 19:29-34; Torimura et al., (2001) Anal. Sci. 17:155-160; Thomas et al. (2002) Pure Appl. Chem. 74:1731-1738; Vullev et al., (2002) Res. Chem. Intermed. 28:95-815; Yamane, A. (2002) Nucleic Acids Research 30: e97; Du et al., (2003) J. Am. Chem. Soc. 125: 4012-4013; Kawai, K., and Majima T. (2003) J. Photochem. Photobiol. C: Photochem. Rev. 3: 53-66; May, et al. (2003) Chem. Comm. 970-971, all of which are incorporated herein by reference in their entireties. All of these quenchers can be incorporated or attached to the capture oligonucleotides of the present invention.

Many embodiments of the present invention afford several significant improvements to standard nucleic acid detection and measurement technology. For example, many capture oligonucleotides of the present invention do not require any chemical modifications for attachment to the substrate surface or for incorporation of a fluorophore, and therefore can be synthesized economically. For another example, many capture oligonucleotides of the present invention can be self-assembled on one or more substrate supports, thereby making the manufacturing of the nucleic acid detector quick and inexpensive. In one embodiment, each one of the thousands of capture oligonucleotides in a large array has the same tail sequence, thereby allowing the use of a single fluorescent reporter oligonucleotide. In another embodiment, the fluorescence quenching output is reversible, and all components of the detection system are immobilized. This allows for real-time in situ nucleic acid detection.

In one aspect, the present invention provides nucleic acid arrays comprising a substrate and a nucleic acid complex. The nucleic acid complex comprises an anchor nucleic acid molecule that is stably attached to the substrate, and an oligonucleotide of the present invention that is hybridized to the anchor nucleic acid molecule. The oligonucleotide comprises (1) a hairpin-forming sequence capable of forming a stem-loop and (2) a reporter-binding sequence capable of hybridizing under nucleic acid array hybridization conditions to a fluorophore-labeled reporter sequence. In many instances, the reporter-binding sequence is complementary to the fluorophores-labeled reporter sequence.

Formation of the stem-loop in the oligonucleotide modifies the fluorescence signals of the fluorophore-labeled reporter sequence when the reporter sequence is hybridized to the oligonucleotide. In many cases, formation of the stem-loop quenches the fluorescence signals of the fluorophore-labeled reporter sequence. For instance, formation of the stem-loop can quench the fluorescence signals of the reporter sequence by at least 10%, 20%, 30%, 40%, 50%, 60%, 70%, 80%, 90% or more, as compared to that when the stem-loop is in an open configuration. In many other cases, disruption of the stem-loop produces a detectable increase in the fluorescence signals of the fluorophore-labeled reporter sequence when the reporter sequence is hybridized to the oligonucleotide. Disruption of the stem-loop can be achieved, for example, by hybridization of the oligonucleotide to a suitable target sequence which forms base-pairing with at least part of the hairpin-forming sequence.

In one embodiment, an oligonucleotide of the present invention is stably associated with a nucleic acid array via hybridizing to an anchor nucleic acid molecule, and the oligonucleotide is also hybridized to a fluorophore-labeled reporter sequence. The oligonucleotide may or may not form the stem-loop or be hybridized to the target sequence. In addition to the use of an anchor nucleic acid molecule, the present invention also contemplates the use of other means for attaching oligonucleotides to nucleic acid arrays, as appreciated by those of ordinary skill in the art.

In one specific example, an oligonucleotide of the present invention comprises at least one guanine base (such as 1, 2, 3, 4, 5, or more guanosines). Formation of the stem-loop in the oligonucleotide brings the guanine base(s) into close proximity to the fluorophore-labeled reporter sequence when the reporter sequence is hybridized to the oligonucleotide, thereby quenching the fluorescence signals of the reporter sequence.

In another specific example, an oligonucleotide of the present invention comprises, from the 3′ end to the 5′ end (or from the 5′ end to the 3′ end), a reporter-binding sequence, a hairpin-forming sequence, one or more guanine base(s), and a sequence capable of hybridizing to an anchor nucleic acid molecule.

Any type of nucleic acid array is contemplated by the present invention, such as traditional microarrays, bead arrays, or microplates. Each of the nucleic acid arrays includes a plurality of discrete regions. The locations of these discrete regions on a nucleic acid array are either predefined or determinable. Each discrete region may be stably associated with an anchor nucleic acid molecule. The anchor molecules in different discrete regions preferably have the same sequence. Anchor molecules with different sequences can also be used.

Each anchor molecule can be hybridized to an oligonucleotide of the present invention. The oligonucleotide in each different discrete region preferably is different, e.g., comprising a different target-binding sequence. The oligonucleotides in different discrete regions can also have the same target-binding sequence. In one embodiment, a nucleic acid array of the present invention includes at least 5, 10, 20, 30, 40, 50, 100, 500, 1,000, or more different capture oligonucleotides of the present invention.

Capture oligonucleotides can also be stably attached to a nucleic acid array without binding to the anchor molecules. These capture oligonucleotides can be attached to different discrete regions on a nucleic acid array via covalent or non-covalent interactions.

The present invention also features nucleic acid complexes comprising an oligonucleotide of the present invention. The oligonucleotide comprises a reporter-binding sequence that is hybridized to a fluorophore-labeled reporter sequence. The oligonucleotide also comprises a hairpin-forming sequence capable of forming a stem-loop. Formation of the stem-loop modifies (e.g., quenches) the fluorescence signals of the reporter sequence. Disruption of the stem-loop (e.g., by hybridizing to a target sequence) can produce a detectable change (e.g., an increase) in the fluorescence signals of the fluorophore-labeled reporter sequence.

In addition, the present invention features methods for detecting the presence or absence of a target sequence. The methods comprise the steps of:

hybridizing an oligonucleotide of the present invention to a nucleic acid sample and a fluorophore-labeled reporter sequence, wherein the oligonucleotide comprises (1) a hairpin-forming sequence capable of forming a stem-loop and (2) a sequence capable of hybridizing under nucleic acid array hybridization conditions to the fluorophore-labeled reporter sequence, wherein the oligonucleotide is capable of hybridization under nucleic acid array hybridization conditions to the target sequence, and hybridization of the oligonucleotide to the target sequence prevents formation of the stem-loop in the oligonucleotide, and wherein formation of the stem-loop quenches fluorescence signals of the fluorophore-labeled reporter sequence when the reporter sequence is hybridized to the oligonucleotide; and

detecting the fluorescent signals of the reporter sequence.

An increase in fluorescence signals of the fluorophore-labeled reporter sequence in the presence of the nucleic acid sample compared to that in the absence of the nucleic acid sample is suggestive of the presence of the target sequence in the sample, while no significant change in fluorescence signals of the fluorophore-labeled reporter sequence in the presence of the nucleic acid sample compared to that in the absence of the nucleic acid sample is suggestive of the absence of the target sequence in the sample.

Furthermore, the present invention features methods for detecting sequence differences between a target sequence and a sequence of interest. The methods comprising the steps of:

hybridizing an oligonucleotide of the present invention to the sequence of interest and a fluorophore-labeled reporter sequence, wherein the oligonucleotide comprises (1) a hairpin-forming sequence capable of forming a stem-loop and (2) a sequence capable of hybridizing under nucleic acid array hybridization conditions to the fluorophore-labeled reporter sequence, wherein the oligonucleotide comprises a sequence that is complementary to the target sequence, and hybridization of the target sequence to the oligonucleotide prevents formation of the stem-loop in the oligonucleotide, and wherein formation of the stem-loop quenches fluorescence signals of the fluorophore-labeled reporter sequence when the reporter sequence is hybridized to the oligonucleotide; and

detecting the fluorescent signals of the reporter sequence.

A decrease in fluorescence signals of the fluorophore-labeled reporter sequence in the presence of the sequence of interest compared to that in the presence of the target sequence (e.g., in the same concentration as the sequence of interest), together with an increase in fluorescence signals of the fluorophore-labeled reporter sequence in the presence of the sequence of interest compared to that in the absence of the sequence of interest, is suggestive that the sequence of interest is homologous to but different from the target sequence. In one example, the sequence difference between the target sequence and the sequence of interest is a single nucleotide mutation. Examples of single nucleotide mutations amenable to the present invention include, but are not limited to, nucleotide substitution, deletion, addition, or another modification that affects base-pairing ability. The present invention also contemplates detection of two or more nucleotide differences between the target sequence and the sequence of interest.

### BRIEF DESCRIPTION OF THE DRAWINGS

The drawings are provided for illustration, not limitation.

FIG. 1 illustrates the components of a nucleic acid detection and measurement system of the present invention.

FIGS. 2A-2C contrast two known forms of G-base quenching (A and B) with a novel G-base quenching of the present invention (C).

FIGS. 3A-3C demonstrate the operation of a nucleic acid detection and measurement system of the present invention. FIG. 3A shows an open configuration of a capture nucleic acid; FIG. 3B indicates hybridization with a target molecule; and FIG. 3C illustrates formation of a stem-loop in the capture nucleic acid.

FIGS. 4A-4C indicate different experimental configurations used to show that G bases on the hairpin loop of the capture oligonucleotide (CO) cause fluorescence quenching of RO-TAMRA.

FIG. 5 shows fluorescence spectra demonstrating that G bases on the hairpin loop of the capture oligonucleotide cause fluorescence quenching of RO-TAMRA.

FIGS. 6A and 6B depict two experimental configurations used to demonstrate that hybridization of a target oligonucleotide traps the capture oligonucleotide in the hairpin-opened form and thus decreases the quenching of RO-TAMRA by the proximal G bases.

FIG. 7 shows fluorescence spectra demonstrating that the effect of a 24mer target on the emission intensities of the RO-CO and RO-CCO hybrids.

FIG. 8 shows the detection of a 24mer target by RO-CO hybrid at room temperature (no premixing or preheating of 24mer with CO).

FIG. 9 illustrates the detection of B7-67mer by RO-CO hybrid at room temperature (no premixing or preheating of B7-67mer with CO).

FIG. 10 describes the effect of the address oligo on quenching.

FIG. 11 illustrates that a single base mismatch between a target oligonucleotide and the sequence in the capture oligonucleotide can be detected as a difference in emission intensity.

### DETAILED DESCRIPTION OF THE INVENTION

### I. Definitions

In order that the present invention may be more readily understood, certain terms are first defined. Additional definitions are set forth throughout the detailed description.

In this application, the use of the singular includes the plural unless specifically stated otherwise. Also, the use of “or” means “and/or” unless state otherwise.

The terms “target nucleic acid” refers to the nucleic acid sequence that is to be detected or measured using the improved methods of the present invention. A target nucleic acid may be deoxyribonucleic acid (DNA), ribonucleic acid (RNA, including messenger ribonucleic acid or mRNA), or other types of nucleic acid molecules.

The term “base pair” refers to a pair of nucleotide bases (nucleotides) each in a separate single stranded nucleic acid in which each base of the pair is non-covalently bonded to the other (e.g., via hydrogen bonds). For instance, a Watson-Crick base pair usually contains one purine and one pyrimidine. Guanosine can pair with cytosine (G-C), adenine can pair with thymine (A-T), and uracil can pair with adenine (U-A). The two bases in a base pair are said to be complementary to each other.

The term “oligonucleotide”, as used herein, refers to a molecule comprised of two or more nucleic acid residues (e.g., deoxyribonucleotides, ribonucleotides or modified forms thereof). Any method can be used to prepare oligonucleotides of the present invention. For instance, oligonucleotides can be synthesized chemically, or expressed from a suitable construct or vector. As used herein, an oligonucleotide can be a polynucleotide and comprise at least 10, 20, 30, 40, 50, or more nucleotide residues.

The terms “hybridization” or “hybridize” include the specific binding of two nucleic acid single strands through complementary base pairing. Hybridization typically involves the formation of hydrogen bonds between nucleotides in one nucleic acid strand and their corresponding nucleotides in the second nucleic acid strand.

The term “melting temperature” (Tₘ) is defined as the temperature at which 50% of the nucleic acid strands in a specific nucleic acid duplex dissociate at a defined ionic strength, pH, and nucleic acid concentration. For hybrids less than 18 base pairs in length, Tₘ(° C.) may be calculated as Tₘ=2(# of A+T bases)+4(# of G+C bases). For hybrids between 18 and 49 base pairs in length, Tₘ(° C.) may be calculated as Tₘ=81.5+16.6(log₁₀Na⁺)+0.41(% G+C)−(600/N), where N is the number of bases in the hybrid, and Na⁺ is the molar concentration of sodium ions in the hybridization buffer.

The terms “hairpin or stem-loop”, as used herein, describe a secondary structure formed by a single-stranded oligonucleotide when complementary bases in one part of the linear strand hybridize with bases in another part of the same strand.

The term “capture oligonucleotide” includes, but is not limited to, a single-stranded sequence of nucleotide bases made up of the following segments of nucleotides progressing from the 3′ terminus to the 5′ terminus (note that this description applies to a sequence attached to a substrate at its 3′ end, but a capture sequence can be similarly prepared for attachment at its 5′ end): 1) a variable length sequence at the 3′ terminus complementary to a particular address oligonucleotide sequence; 2) a sequence of guanosine bases positioned just 3′ of the hairpin or stem-loop sequence; 4) a sequence of bases of variable length complementary to a sequence in the nucleic acid that is to be detected and measured, 5) a sequence that is complementary to the first 5 to 15 bases in the nucleic acid recognition sequence (note that this can include probes that use exclusively the loop region as the nucleic acid recognition sequence), and that upon hybridization forms a hairpin or stem-loop secondary structure; and 6) a sequence of bases of variable length ending at the 5′ terminus that are complementary to the sequence of a fluorophore-labeled reporter oligonucleotide. Heating the hairpin-forming sequence to its melting temperature or hybridization with the target nucleic acid can linearize the capture oligonucleotide. Each functional segment in the above-described capture molecule can be re-arranged as desired. In addition, the guanosine bases can be replaced by other naturally occurring, modified or synthetic bases, provided that desirable fluorescence quenching can be achieved. Other quenching moieties can also be employed in the capture molecule.

The term “address oligonucleotide” includes, but is not limited to, a single-stranded sequence of nucleotide bases derivatized on either its 5′ or 3′ end with a functional group capable of forming a covalent bond with a functional group on a substrate. For the purpose of illustration only, the functional group on the address oligonucleotide could be an amino group and the functional group on the substrate could be a carboxyl group, thus enabling the formation of an amide linkage. The address oligonucleotide has a base sequence that is complementary to a base sequence at either the 5′ or 3′ terminus of the capture oligonucleotide. Hybridization of the capture oligonucleotide with the surface-immobilized address oligonucleotide results in the tethering of the capture oligonucleotide to the substrate. Microarrays with a universal set of address sequences can be used for any targets simply by controlling the combination of the sequences of the address-binding region and the target-binding region of the capture oligonucleotide. Also, the length and number of complementary bases in the address oligonucleotide can be varied to affect the desired strength of the tether (melting temperature).

The term “self-assembly” as used herein refers to the attachment of the capture oligonucleotide to the surface substrate by hybridization with the address oligonucleotide, and also to the attachment of the reporter oligonucleotide to the capture oligonucleotide by hybridization.

The term “guanosine bases” refers to one or more guanosine nucleotides in either a single-stranded nucleic acid sequence, or in a double-stranded nucleic acid sequence in which the guanosine bases are base paired with cytosine bases.

The term “G-base quenching” describes the reduction in fluorescence emission of a fluorophore when in close proximity to guanosine bases in the sequence of a single or double-stranded nucleic acid.

The phrase “target nucleic acid recognition sequence” represents the single-stranded sequence within the capture oligonucleotide that is complementary to a sequence in a target nucleic acid. The target nucleic acid recognition sequence can include any portion of the sequence of the loop or one arm of the stem of the capture oligonucleotide. The target nucleic acid recognition sequence can also be exclusively the sequence of the loop. In the case of MnRNA, the sequence would be complementary to a sequence in the single-stranded MnRNA.

The term “hairpin-forming sequence” refers to a sequence in the capture oligonucleotide that can form a hairpin structure. In one specific example, the hairpin-forming sequence is adjacent to, overlaps or includes the target nucleic acid recognition sequence.

The term “quench” means a relative reduction in the fluorescence intensity of a fluorescent group as measured at a specified wavelength as well as the complete reduction, regardless of the mechanism by which the relative reduction is achieved. As specific examples, the quenching may be due to molecular collision, energy transfer such as FRET, a change in the fluorescence spectrum (color) of the fluorescent group or any other mechanism. The amount of the relative reduction is not critical and may vary over a broad range. The only requirement is that the reduction be reliably measurable by the detection system being used. Thus, a fluorescence signal is “quenched” if its intensity at a specified wavelength is reliably reduced by any measurable amount. The reduction can be, for example, at least 10%, 20%, 30%, 40%, 50%, 60%, 70%, 80%, 90%, even 100%, as compared to the original fluorescent intensity.

The phrase “stably attached,” means that an oligonucleotide maintains its relative position on a substrate during hybridization and subsequent signal detection. An oligonucleotide can be stably attached to a substrate by non-covalent or covalent interactions.

The phrase “nucleic acid array hybridization conditions” are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, high stringent nucleic acid array hybridization conditions are selected to be about 5-10° C. lower than the Tₘ for the specific sequence at a defined ionic strength pH. Low stringent nucleic acid array hybridization conditions are generally selected to be about 15-30° C. below the Tₘ. Typically, nucleic acid array hybridization conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Nucleic acid array conditions can also include the use of destabilizing agents such as formamide. For selective or specific hybridization, a positive signal preferably is at least two times background, and more preferably, is at least 10 times background.

### II. The Invention

In one aspect, the present invention features a nucleic acid complex which comprises a capture oligonucleotide hybridized to a fluorophore-labeled probe sequence. The capture oligonucleotide comprises a hairpin-forming sequence and is capable of hybridizing under nucleic acid array hybridization conditions to a target sequence. The hairpin formation by the hairpin-forming sequence can modify the fluorescence signals of the probe sequence. Hybridization of the target sequence to the capture oligonucleotide disrupts the hairpin formation, thereby producing a detectable change in the fluorescence of the probe sequence. The detectable change may be a change in any fluorescence property, such as intensity, maximum emission or excitation wavelength, or fluorescence decay property. The modification of the fluorescence signals by the hairpin structure can be, for example, G-base quenching or any other fluorescence property modification.

In one specific example, the nucleic acid complex includes both the target and probe sequences hybridized to the capture oligonucleotide.

In another aspect, the present invention features a nucleic acid array which includes a nucleic acid complex. The nucleic acid complex includes a capture oligonucleotide hybridized to an anchoring sequence stably attached to a substrate of the nucleic acid array. The capture oligonucleotide also includes a hairpin-forming sequence and is capable of hybridizing under nucleic acid array hybridization conditions to a target sequence and a fluorophore-labeled probe sequence Concurrent hairpin formation by the hairpin-forming sequence and hybridization of the probe sequence to the capture oligonucleotide modify the fluorescence signals of the probe sequence. Hybridization of the target sequence to the capture oligonucleotide disrupts the hairpin formation, thereby producing a detectable change in the fluorescence signals of the probe sequence.

The present invention contemplates any type of nucleic acid array, including bead arrays in which nucleic acid complexes are stably attached to numerous beads. The substrate of the nucleic acid array can be made of any material, such as glasses, silica, ceramics, nylon, quartz wafers, gels, metals, and paper.

In one specific example, the nucleic acid complex on the nucleic acid array includes both the target and probe sequences hybridized to the capture oligonucleotide. In another specific example, the nucleic acid complex includes the probe sequence hybridized to the capture oligonucleotide. The nucleic acid complex also comprises a hairpin structure formed by the hairpin-forming sequence. The fluorescence signals of the probe sequence are quenched due to the formation of the hairpin structure.

In yet another aspect, the present invention features a method useful for detecting or measuring a target sequence of interest. The method includes the steps of hybridizing a capture oligonucleotide to a nucleic acid sample and a fluorophore-labeled probe sequence, and detecting the fluorescent signals of the probe sequence. The capture oligonucleotide includes a hairpin-forming sequence and is capable of hybridizing under nucleic acid array hybridization conditions to the target sequence. Concurrent hairpin formation by the hairpin-forming sequence and hybridization of the probe sequence to the capture oligonucleotide modify the fluorescence signals of the probe sequence. Hybridization of the target sequence to the capture oligonucleotide disrupts the hairpin formation, thereby producing a detectable change in the fluorescent signals of the probe sequence.

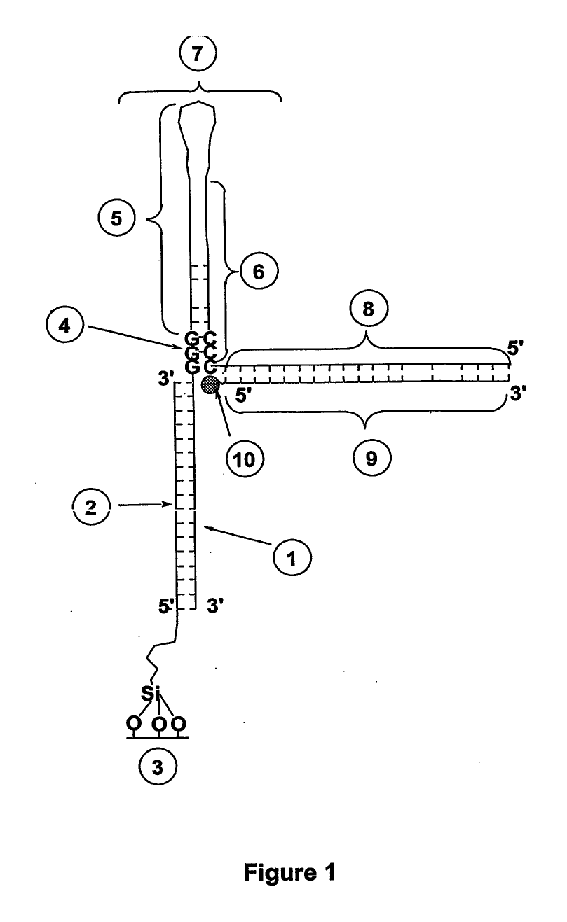

In one embodiment for detecting and measuring the class of nucleic acids known as mRNA (for example only as one class of nucleic acids that can be detected by the method of the present invention), a hairpin or stem-loop structure of capture oligonucleotide is synthesized using standard nucleic acid synthesis techniques. Other techniques like polymerase chain reaction are known in the art and can be used to manufacture the capture oligonucleotide sequence. Although a single sequence is described, it is understood that thousands of these sequences can be made and tested simultaneously in a gene expression array. The components of the improved method are illustrated in FIG. 1. For illustrative purposes, a single-stranded DNA oligonucleotide is shown, the length of which can vary depending on the requirements for detection as will become apparent in the following description. Beginning at its 3′ terminus, the capture oligonucleotide 1 has a sequence of variable length that is complementary to a single-stranded address oligonucleotide 2 that is attached to a substrate surface 3 such as glass or gold-coated silica. Watson-Crick base-pairing or hybridization of these two sequences results in the attachment or self-assembly of the capture oligonucleotide 1 to the substrate surface 3. Continuing in a 5′ direction, the next required sequence is a series of guanosine (G) bases 4 in the positions indicated. The need for these guanosine nucleotides will be described below. Next in the 5′ direction is a sequence of nucleotides that are complementary to a sequence in the mRNA of the gene to be measured. This mRNA recognition sequence 5 can be shorter or longer than shown in the illustration. Next is a sequence of nucleotides complementary to nucleotides in the mRNA recognition sequence. In this hairpin-forming sequence 6, the number of complementary bases can be shorter or longer than illustrated. Hybridization of these bases to their complementary bases results in the formation of a secondary structure called a hairpin or stem-loop 7. Finally, it can be seen in FIG. 1 that there is a “tail” structure consisting of unpaired nucleotides that form the 5′ terminus of the capture oligonucleotide sequence. This 5′ tail sequence 8 can be shorter or longer than that illustrated. The sequence of these bases is complementary to an oligonucleotide sequence called the reporter oligonucleotide 9. From the illustration, it can be seen that the reporter oligonucleotide has a fluorophore 10 attached at its 5′ end.

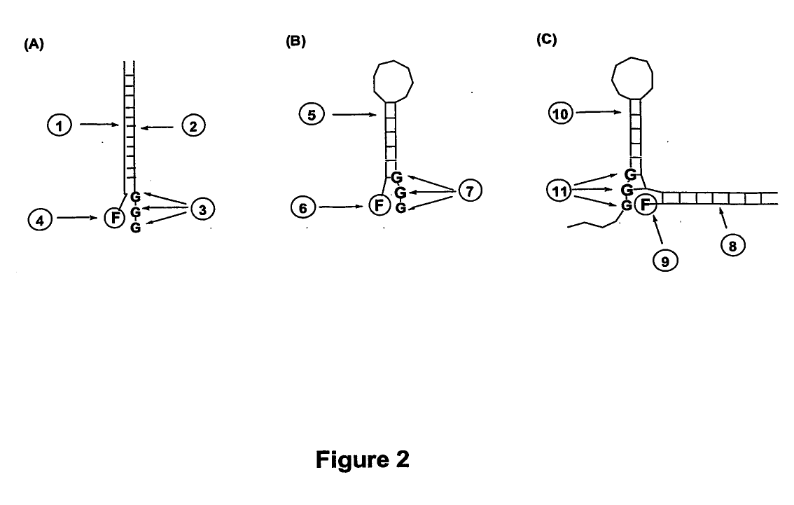

Certain fluorophores chemically attached to oligonucleotide strands will exhibit a characteristic fluorescence emission when excited by light at a characteristic wavelength, and that this characteristic fluorescence emission is significantly reduced when these single-stranded oligonucleotides hybridize to complementary single-strands that have one or more guanosine bases in the vicinity of the fluorophore (see e.g., Morrison et al. (1989) Anal Biochem 183:231-244; Seidel et al. (1996) J Phys Chem 100:5541-5553; Broude et al. (2001) Nucl Acids Res 29:No. 19 e92; Kurata et al. (2001) Nucl Acids Res 29:No. 6 e34). Also see the following U.S. Patents Livak et al. U.S. Pat. No. 5,723,591, Nardone et al. U.S. Pat No. 6,117,986, Livak et al. U.S. Pat No. 6,258,569, and Hawkins, U.S. Pat. No. 6,451,530. The reduction in fluorescence emission when the fluorophore is in close proximity to the guanosine bases is known as G-base quenching and has been described in detail in the scientific literature, see e.g. Torimura et al., (2001) Anal Sci 17:155-160; Zahavy and Fox (1999), J Phys Chem B 103:9321-9327; Crockett and Wittwer (2001), Anal Biochem 290:89-97. In this configuration (FIG. 2A), first strand 1 is base-paired to second strand 2, and second strand 2 has a series of guanosine (G) bases 3 that are in close proximity to fluorophore 4 when strands 1 and 2 are base-paired. Similarly, fluorescence emission of a fluorophore can be quenched (FIG. 2B) if a single-stranded oligonucleotide forms a hairpin or stem-loop configuration 5 that brings fluorophore 6 attached to a base at one end of the strand into close proximity to guanosine bases 7 on the other end of the same strand, see e.g. Walter and Burke (1997) RNA 3:392-404. However, in the method of the invention (FIG. 2C), we have discovered that a reporter oligonucleotide 8 with an attached fluorophore 9 hybridized to a single-stranded oligonucleotide with the potential of forming a hairpin or stem-loop configuration 10 will have its fluorescence emission quenched if there are guanosine bases 11 in the vicinity of the fluorophore 9 when the structure is in the hairpin or stem-loop configuration 10.

The invention allows an oligonucleotide sequence to be quickly and inexpensively labeled with a fluorophore, and obviates the need to chemically label a longer sequence with a fluorophore. Labeling of longer sequences is more difficult and requires more expensive and time-consuming purification procedures. Because the same 5′ tail sequence complementary to the reporter oligonucleotide can be added to each capture oligonucleotide, only a single fluorophore-labeled reporter oligonucleotide needs to be manufactured in order to detect tens of thousands of gene sequences in an array. The benefit of only labeling one nucleotide sequence with a fluorophore should be apparent Moreover, the attachment of the reporter oligonucleotide to the capture oligonucleotide, as was seen in the attachment of the capture oligonucleotide to the address sequence, is done through self-assembly, thus making the addition of a fluorophore to the capture oligonucleotide as simple as mixing the reporter and capture oligonucleotides together under conditions that allow hybridization.

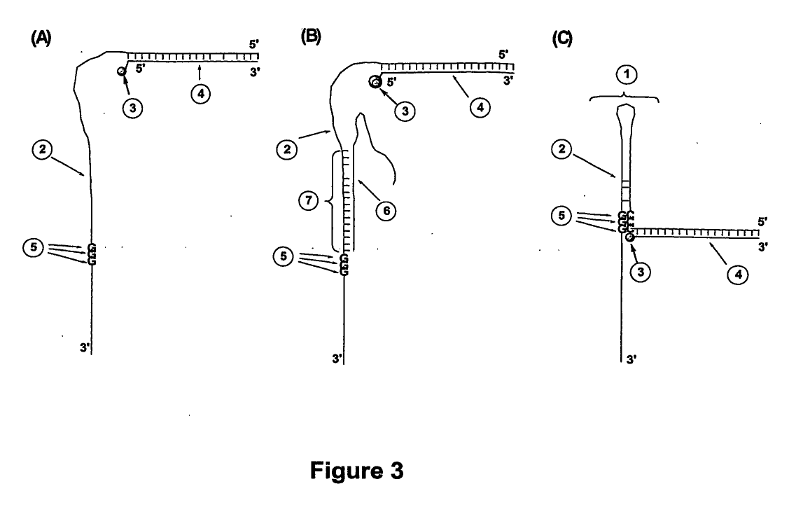

In one mode of operating the present invention, the hairpin or stem-loop configuration 1 of capture oligonucleotide 2 is heated to a temperature that causes the secondary structure to linearize (FIG. 3A). When this occurs, the fluorophore 3 on the reporter oligonucleotide 4 is no longer in close proximity to the guanosine bases 5, and thus its fluorescence is no longer quenched. If a nucleic acid strand like a mRNA 6 that bears a sequence complementary to the mRNA recognition sequence 7 in the capture oligonucleotide 2 is added to the heated capture oligonucleotide (in the open configuration), and then the system is allowed to cool, the mRNA 6 will hybridize with the capture oligonucleotide 2 thus preventing the formation of the hairpin or stem-loop 1 secondary structure (FIG. 3B). This prevents the quenching of the fluorophore 3 on the reporter oligonucleotide 4. If on the other hand, the complementary mRNA sequence is not present in the test sample (FIG. 3C), the hairpin or stem-loop configuration 1 of the capture oligonucleotide 2 will reform upon cooling and the fluorophore 3 on the reporter oligonucleotide 4 will once again be in close proximity to the guanosine bases 5 on the capture oligonucleotide 2, and thus its fluorescence will be quenched. Therefore, the presence of a target nucleic acid in a biological sample is indicated by an inhibition of fluorescence quenching.

A further advantage of the configuration is the ability to generate internal references of fluorescent intensity in order to mathematically estimate target nucleic acid concentrations. In the absence of target nucleic acid, fluorescent intensity can be measured with all hairpin or stem-loop structures in the quenched state by cooling, generating a closed-configuration reference signal. In the presence or absence of target nucleic acid, fluorescence intensity can be measured when all hairpin or stem-loop structures are in the open state by heating to a temperature that causes the secondary structure to linearize (FIG. 3A), generating an open-configuration reference signal. Either or both of these fluorescent intensities can be used as reference signals for comparison to determine the presence of target nucleic acid in a test sample. Fluorescent intensities approximately equal to the closed-configuration reference signal indicate the absence or very low concentrations of target nucleic acid. Increasing fluorescent intensities, approaching the open-configuration reference signal, indicate increasing concentrations of target nucleic acid.

Because a method of the invention detects and measures nucleic acid sequences using self-assembly through hybridization, one of the design considerations involves the temperature at which each set of hybridized oligonucleotides dissociates or melts (melting temperature or Tₘ). Although the specific sequence of bases in the address, hairpin, and reporter oligonucleotides can be varied, the mixture of A, T, C, and G bases may be preferred as it determines the temperature at which two base-paired strands dissociate or melt. In particular, Tₘ of double stranded oligonucleotides is influenced by the relative numbers of G and C bases generally according to the formula Tₘ=69° C.+0.41 (molar % G-C)-650/average length of probe. The dependence of Tₘ of the stem region of the hairpin on the base sequence can be predicted from the free energy of formation of the stem hybrid calculated using DNA folding program such as the Zuker folding program. Although it is not necessary in a method of the invention to open the hairpin by heating before hybridization with target nucleic acid, the linearization of the hairpin by heating will facilitate hybridization with target. Therefore, it is preferred that the melting temperatures of the address oligonucleotide and reporter oligonucleotide to their respective complementary sequences in the capture oligonucleotide are higher than the temperature used to melt the hairpin. In addition, it is advantageous to have the melting temperature of the target nucleic acid strand with the nucleic acid recognition sequence higher than the melting temperature of the hairpin-forming sequence hybridized to the hairpin sequence. This facilitates the capture of the target nucleic acid by allowing it to hybridize to the target nucleic acid recognition sequence at a temperature that maintains the hairpin in the open configuration. Even a 10° C. difference in melting temperatures is more than sufficient to allow the melting of the hairpin structure without the release of the capture oligonucleotide from the address oligonucleotide or the release of the reporter oligonucleotide from its complementary sequence on the tail of the capture oligonucleotide. Techniques for thermocycling with precise temperature control are well known to those skilled in the art. Moreover, one skilled in the art through the use a variety of commercially available and free software programs for designing nucleotide probes can easily accomplish calculation of melting temperatures.

The present invention will be more clearly understood from the following specific examples. These examples are provided solely for illustrative purposes and should not be construed to limit the scope of the invention in any way.

### EXAMPLES

To illustrate the operation of the present invention, several studies were performed in which a specific nucleic acid sequence was detected in solution using a hairpin capture oligonucleotide to which was attached a fluorophore-labeled reporter oligonucleotide. The nucleic acid sequences detected were all from the murine B7.2 gene; see GenBank BC613807, GI:15489434.

### General Materials and Methods

The following oligonucleotides were custom synthesized by Integrated DNA Technologies, Inc. (IDT, Coralville, Iowa) or Synthetic Genetics, Inc. (San Diego, Calif.). The base sequences were designed with the aid of OligoAnalyzer 3.0 software from IDT to achieve specific melting temperatures, and to minimize the formation of self-dimers, unwanted hairpins, and cross-hybridization. Note that all sequences are given in the 5′->3′ orientation. One or more of the following oligonucleotides was used in the examples one through five:

Reporter Oligonucleotide (RO-TAMRA): TAMRA-AAAATCCACCCACCCCACCC (SEQ ID NO:1). This 5′-TAMRA-labeled oligonucleotide is complementary to the 5′ tail sequence of the capture oligonucleotide.

Reporter Complement (RC): GGGTGGGGTGGGTGGATTTT (SEQ ID NO:2). This oligonucleotide is complementary to the reporter oligonucleotide, and was used to determine if a G base five nucleotides away from the TAMRA fluorophore could cause quenching.

Capture Oligonucleotide (CO): GGGTGGGGTGGGTGGATTTTCCCAAACTTACGGATCGTGGGTGCTTCCGTAA GTTTGGGCCCCTCCTCCTCCCTCCTCC (SEQ ID NO:3). This 79-mer oligonucleotide has a short nucleotide sequence complementary to a sequence in the murine B7.2 mRNA.

Control Capture Oligonucleotide (CCO): GGGTGGGGTGGGTGGATTTTAAAAAACTTACGGATCGTGGGTGCTTCCGTAA GTTTTTTCCCCTCCTCCTCCCTOCTCC (SEQ ID NO:4). This oligonucleotide has the same sequence as the capture oligonucleotide except that three thymines replace three guanines at positions 23 to 25 (from the 5′ terminus).

24mer Target Sequence (24mer): CCCAAACTTACGGAAGCACCCACG (SEQ ID NO:5). This oligonucleotide represents a target that is complementary to 24 nucleotides in the target recognition sequence in the CO and CCO.

B7-67mer Target Sequence (B7-67mer). CCAGAACTTACGGAAGCACCCACGATGGACCCCAGATGCACCATGGGCTTG GCAATCCTTATCTTG (SEQ ID NO:6). This oligonucleotide represents a segment of the murine B7.2 mRNA sequence. Its sequence is complementary to the 22 nucleotides in the mRNA recognition sequence.

Address Oligonucleotide with Disufide (AO/SS): 5′-disulfide-GGAGGAGGGAGGAGGAGGGG (SEQ ID NO:7). This oligonucleotide has a disulfide group at the 5′ end that enables its attachment to the substrate. Hybridization of the capture oligonucleotide to this address oligonucleotide results in the surface attachment of the capture sequence.

### Preparation of Nucleic Acid Samples

The oligonucleotides were dissolved in TE buffer (Tris-EDTA buffer: 10 mM Tris-HCl, 1 mM EDTA, 1 M NaCl, ˜pH 7.7). The TE buffer solutions were prepared with doubly distilled water (Barnstead MegaPure 3 system) and filtered with a sterile, 0.2 μm nylon syringe filter (Nalgene™) before used.

### Fluorescence Spectroscopy

All fluorescence spectra were collected with a Spex Fluorolog 3 fluorescence spectrometer (Instrument S.A., Inc., New Jersey). Both excitation and emission monochromators utilize double mechanically blazed planar gratings. Emissions from solutions in cuvettes were collected at 90° with respect to the incident light. The samples were excited at 555 nm and the emission spectra were collected in one single scan in the wavelength range of 570-675 nm (with an increment of 1 nm and integration time of 0.5 s).

Variable temperature experiments were performed using a single cell sample heater/cooler holder (model FL 1027, JY Inc., New Jersey). The temperature of the sample holder was varied by circulating water from a temperature-controlled water bath (Fisher Scientific Model #9150). After the desired temperature was attained, a cuvette containing the sample was placed in the jacketed sample holder and the solution was equilibrated for 5 minutes. Longer equilibration time was avoided to minimize evaporation of solvent. The difference in the actual sample temperature from the temperature readout of the circulator was calibrated as follows. After the desired temperature of the water circulator was attained, a TE buffer solution in a cuvette was placed in the sample holder to equilibrate for 5 minutes. The actual temperature of the buffer solution and the temperature of the circulating water in the circulator were measured using a thermometer and compared to the temperature read out of the circulating bath. The temperature values for all experiments described below were the corrected temperatures of the samples.

### Example 1

### Evidence for the Fluorescence Quenching of RO-TAMRA by G Bases on the Hairpin Loop of the Capture Oligonucleotide

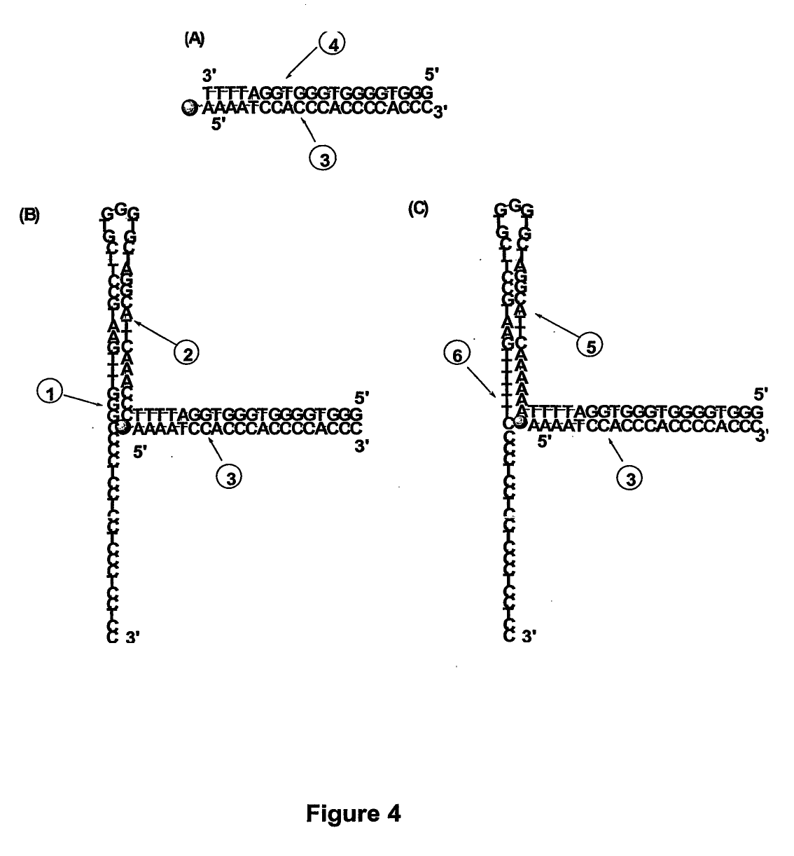

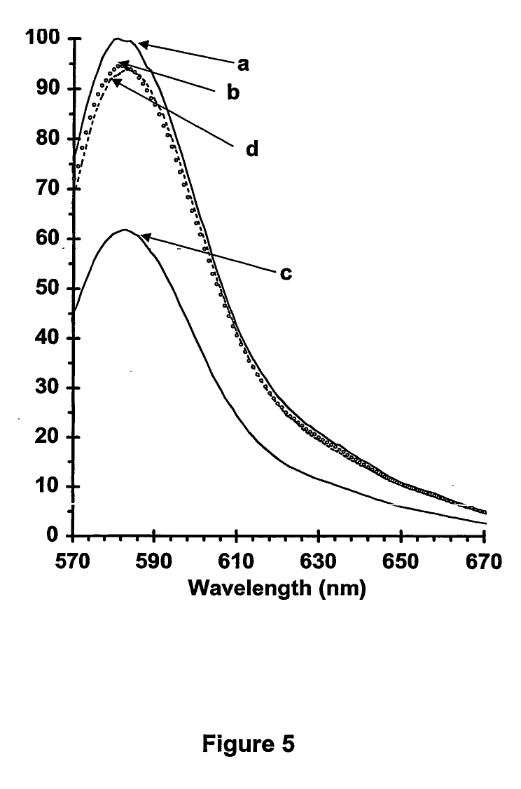

To evaluate the effectiveness of the G-bases 1 (shown in FIG. 4B) in the hairpin loop of CO 2 in quenching the emission of RO-TAMRA 3, the changes in fluorescent emission of RO-TAMRA 3 upon hybridization with RC 4 (FIG. 4A), CO 2 (FIG. 4B), and CCO 5 (FIG. 4C), respectively, were compared. Three aliquots 1-3 (600 μL each) of a 1.5×10⁻⁶ M solution of RO-TAMRA were prepared and their fluorescent emission spectra recorded. Small volumes of the solutions (˜8.8×10⁻⁴ M in concentration) of RC (2 μL), CO (˜1.1 μL), and CCO (1 μL) were added to solutions 1-3, respectively. The fluorescent emission spectra of the resultant solutions were recorded at 25° C. To facilitate the comparison of the fluorescence intensities of different solutions, all emission spectra were normalized. The maximum emission intensity of each solution of RO-TAMRA before the addition of other oligonucleotides was considered as 100% (FIG. 5a). The relative emission intensities of the solutions after the addition of RC, CO, or CCO with respect to the maximum emission intensity before the addition of RC, CO, or CCO were calculated.

As shown in FIG. 5b, a small decrease (˜6%) in emission intensity of RO-TAMRA was observed upon hybridization with RC. This moderate quenching may have been due to the presence of a G base five nucleotides away from TAMRA, or perhaps due to the moderate quenching effects of other nucleotides in the RC sequence. On the other hand, a much larger decrease (˜40%) in emission intensity of RO-TAMRA was observed upon hybridization with CO in the hairpin-closed form (FIG. 5c). These results strongly indicated that G bases on the hairpin loop segment of CO quenched TAMRA fluorescence. However, to verify the G base quenching, we designed CCO in which three G bases were replaced by three T bases 6 as shown in FIG. 4C. Hybridization of RO-TAMRA with CCO resulted in only about 6% fluorescence quenching of TAMRA (FIG. 5d), similar in magnitude to the quenching by RC. This result indicated that the G bases on the closed hairpin loop that were in proximity to TAMRA mainly caused the large quenching effect of CO.

### Example 2

### Detection of 24mer Target Oligonucleotides by Hybridization with CO in the Hairpin-Opened Form

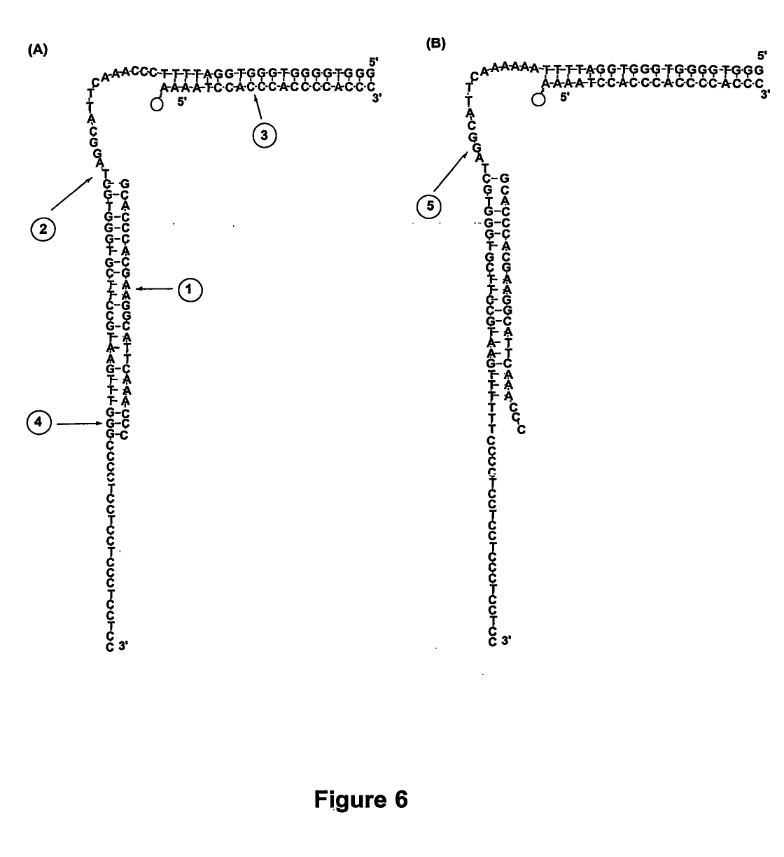

A 24-mer strand (24mer) complementary to the mRNA recognition sequence of the CO was used to demonstrate that the hybridization of target oligonucleotide 1 traps the CO 2 in the hairpin-opened form (FIG. 6A) and thus decreases the quenching of RO-TAMRA 3 by the G bases 4 in the hairpin section. Control experiments were performed using CCO 5 (FIG. 6B) instead of CO 2.

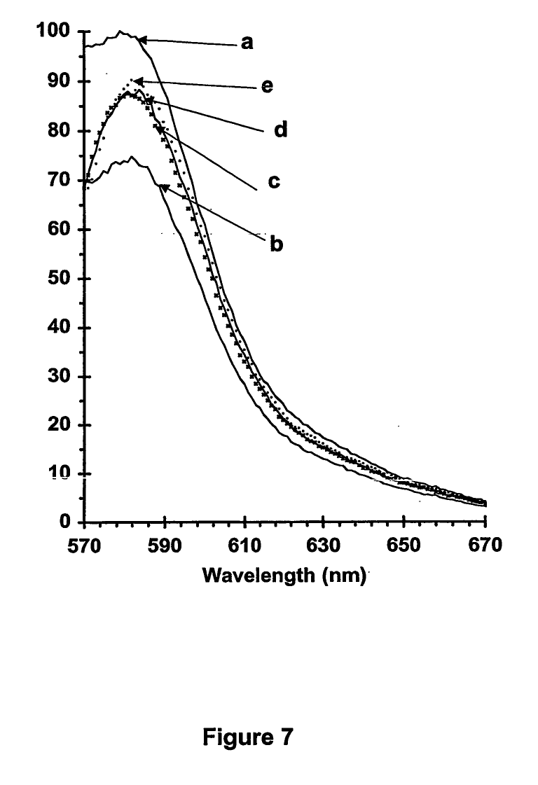

The solutions listed in Table 1 were prepared. Solutions 6 and 8 were heated to 76° C. for 10 min to open the hairpins and then cooled to 25° C. to allow hybridization with the target 24mer. After the fluorescent emissions from solutions 1-4 were recorded, 2-μL aliquots of solutions 5-8 were added to solutions 1-4 respectively to give solutions 1a-4a. The fluorescence emissions from solutions 1a-4a were then recorded. The maximum emission intensity of the solutions 1-4 before the addition of other oligonucleotides was considered as 100 (FIG. 7a). The relative emission intensities of the solutions 1a-4a with respect to the maximum emission intensity before the addition of RC, CO, or CCO were calculated.

TABLE 1
Solutions used for studying the effect of target 24mer
on the emission intensity of the RO-CO hybrid

| | Composition | | | |
|---|---|---|---|---|
| Solution # | RO | CO | CCO | 24mer |
| 1-4 (600 μL | 1.5 × 10⁻⁸ M | 0 | 0 | 0 |
| each) | | | | |
| 5 | 0 | 9.7 × 10⁻⁶ M | 0 | 0 |
| 6 | 0 | 4.9 × 10⁻⁶ M | 0 | 1.0 × 10⁻³ M |
| 7 | 0 | 0 | 9.7 × 10⁻⁶ M | 0 |
| 8 | 0 | 0 | 4.9 × 10⁻⁶ M | 1.0 × 10⁻³ M |
| 1a (2 μL of | 1.5 × 10⁻⁸ M | 3.2 × 10⁻⁸ M | 0 | 0 |
| 5 added to 1) | | | | |
| 2a (2 μL of | 1.5 × 10⁻⁸ M | 1.6 × 10⁻⁸ M | 0 | 3.3 × 10⁻⁶ M |
| 6 added to 2) | | | | |
| 3a (2 μL of | 1.5 × 10⁻⁸ M | 0 | 3.2 × 10⁻⁸ M | 0 |
| 7 added to 3) | | | | |
| 4a (2 μL of | 1.5 × 10⁻⁸ M | 0 | 1.6 × 10⁻⁸ M | 3.3 × 10⁻⁶ M |
| 8 added to 4) | | | | |

As shown in FIG. 7 and Table 2, the hybridization of CO in the closed hairpin form to RO-TAMRA led to significant quenching (˜25%) of the TAMRA emission by the G bases in the hairpin section of the CO (FIG. 7b). Prehybridization of the target 24mer with CO trapped the hairpin in the open form. As a consequence, the hybridization of this opened hairpin with RO-TAMRA resulted in a much weaker quenching (˜13%) of the TAMRA emission (FIG. 7c). As expected, the intensity of emission from the hairpin-opened RO-TAMRA-CO-24mer hybrid (FIG. 7c) was similar to the emissions from the RO-TAMRA-CCO hybrid (FIG. 7d) and the RO-TAMRA-CCO-24mer hybrid (FIG. 7e) since all three hybrids were only quenched by the G-bases in the sequence complementary to RO-TAMRA. It should be noted that in the absence of CO or CCO, the addition of excess 24mer to RO did not cause observable change in the fluorescent emission of TAMRA. This result confirmed that there was no direct influence of 24mer on the fluorescent emission of RO.

TABLE 2
Summary of the studies on the effect of target 24mer on
the emission intensities of the RO-CO and RO-CCO hybrids

| | Percent Decrease in |
|---|---|
| Solution | Emission Intensity (%) |
| 1 (RO only) | 0% |
| 1a (RO + CO) | 25% |
| 2 (RO only) | 0% |
| 2a (RO + CO + 24mer) | 13% |
| 3 (RO only) | 0% |
| 3a (RO + CCO) | 12% |
| 4 (RO only) | 0% |
| 4a (RO + CCO + 24mer) | 10% |
| RO + 24mer | 0% |

### Example 3

### Detection of 24mer Target Oligonucleotides by Hybridization with CO Without Preheating CO to the Hairpin Opened Form

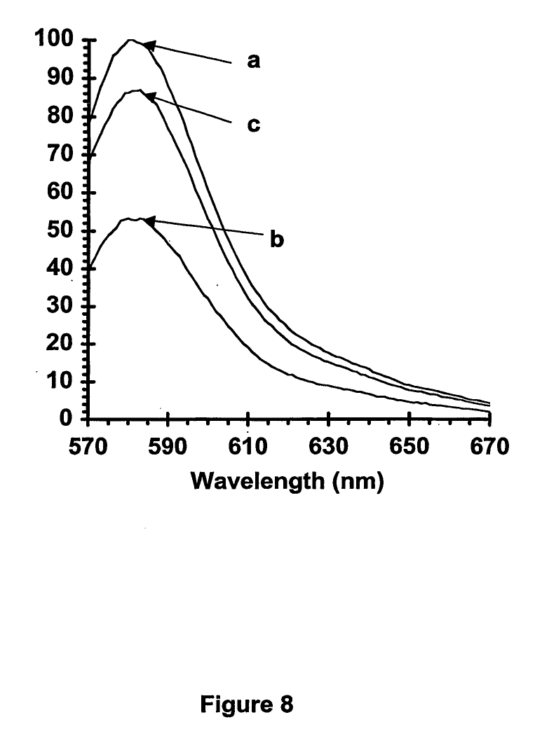

This example illustrates an alternative procedure for detecting target nucleic acid without preheating the capture oligonucleotide to the hairpin opened form and prehybridization of the target with the hairpin opened capture oligonucleotide. In this example, 600-μL of a ˜1.7×10⁻⁷ M solution of RO-TAMRA was prepared and the fluorescent emission spectrum of the solution was recorded (FIG. 8a). Small volumes of a solution (˜1.0×10⁻⁴ M in concentration) of CO were added to the solution of RO-TAMRA until no further decreased in fluorescence intensity of the solution was observed. A small volume (3 μL) of a solution (˜9.2×10⁻⁵ M) of target 24mer was then added and allowed to hybridize with the RO-CO hybrid. The concentrations RO-TAMRA, CO, and 24mer target in the resultant solution were approximately 1.7×10⁻⁷ M, 3.4×10⁻⁷ M, and 4.6×10⁻⁷ M, respectively. The change in fluorescence intensity was monitored. As shown in FIG. 8 and Table 3, after the addition of 2 μL of CO to hybridized RO-TAMRA, a large decrease (˜45%) in emission intensity of RO-TAMRA was observed (FIG. 8b). Hybridization of 24mer with RO-CO trapped the capture oligonucleotide in the hairpin-opened form, and thus reduced the quenching of TAMRA emission and resulted in an increase in emission intensity by ˜30% (FIG. 8c).

TABLE 3
Summary of the effect of target 24mer on the
emission intensities of the RO-CO hybrids

| | Percent Decrease in |
|---|---|
| Solution | Emission Intensity (%) |
| RO only | 0% |
| RO + CO | ~45% |
| RO + CO + 24mer | ~15% |

### Example 4

### Detection of B7-67mer Target Oligonucleotides by Hybridization with CO in the Hairpin-Opened Form

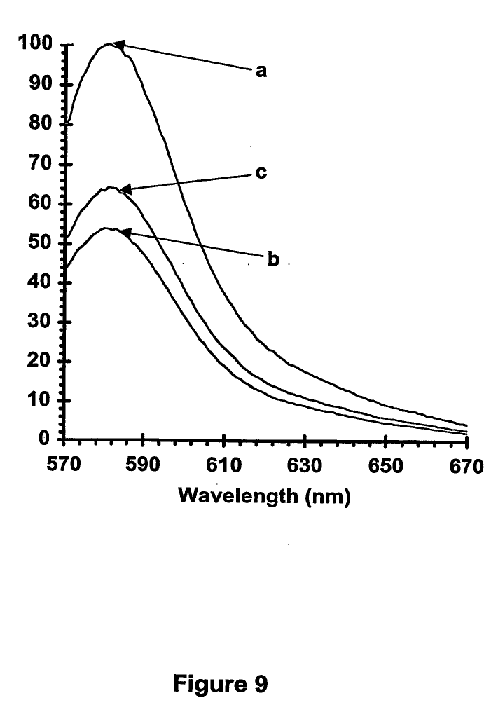

In this example, 600-μL of a 1.7×10⁻⁷ M solution of RO-TAMRA was prepared and the fluorescent emission spectrum of the solution was recorded (FIG. 9a). Small volumes of a solution (1.0×10⁻⁴ M in concentration) of CO were added to the solution of RO-TAMRA until no further decreased in fluorescence intensity of the solution was observed. A small volume (3 μL) of a solution (9.2×10⁻⁵ M) of target B7-67 mer was then added and allowed to hybridize with the RO-CO hybrid. The change in fluorescence intensity was monitored. As shown in FIG. 9 and Table 4, after the addition of 2 μL of CO to hybridized RO-TAMRA, a large decrease (˜45%) in emission intensity of RO-TAMRA was observed (FIG. 9b). Hybridization of B7-67mer with RO-CO trapped the capture oligonucleotide in the hairpin-opened form, and thus reduced the quenching of TAMRA emission and resulted in an increase in emission intensity by ˜10% (FIG. 9c). No change in emission intensity of TAMRA was observed when B7-67mer was added to a solution of RO-TAMRA in the absence of CO. This confirms that there was no direct influence of B7-67mer on the fluorescent emission of RO-TAMRA (Table 4).

TABLE 4
Summary of the effect of target B7-67mer on the
emission intensities of the RO-CO hybrids.

| | Percent Decrease in |
|---|---|
| Solution | Emission Intensity (%) |
| RO only | 0% |
| RO + CO | ~45% |
| RO + CO + B7-24mer | ~35% |

### Example 5

### Effect of AO-SS on the Emission Intensities of the RO-TAMRA-CO and RO-TAMRA-CCO Hybrids

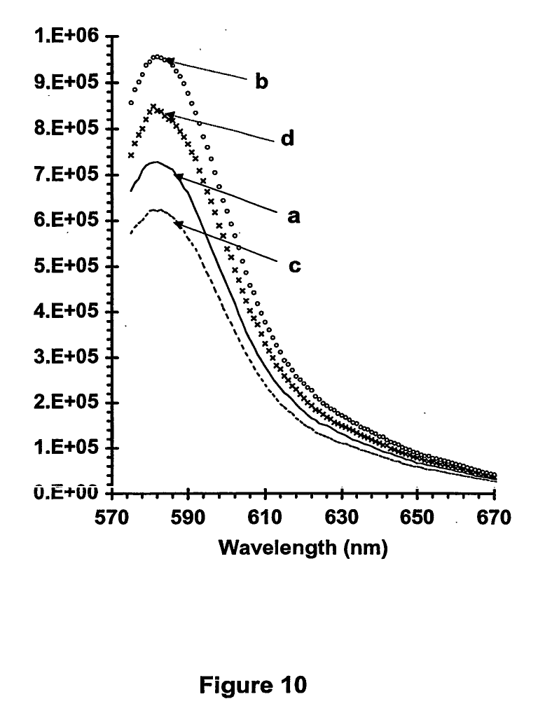

Further quenching of TAMRA in RO-TAMRA-CO by hybridization with an address oligonucleotide that is rich in G bases at the 3′ end could maximize the difference in emission intensity between the hairpin-closed form and the opened form upon hybridization with target strand. In this example, solutions of RO-TAMRA (1.7×10⁻⁷ M) hybridized with CO (2.0×10⁻⁷ M) or CCO (2.0×10⁻⁷ M) in TE buffer were prepared. The fluorescence emission spectra of these solutions containing the RO-CO and RO-CCO were shown in FIGS. 9a and 9b, respectively. To each solution was then added 1.2 uL of a 1.0×10⁻³ M solution of AO-SS. The concentration of AO-SS in the resultant solutions was ˜2.0×10⁻⁶ M. As shown in FIG. 10 and Table 5, the hybridization of the RO-TAMRA-CO hybrid with AO-SS decreased the fluorescent emission of RO-CO by about 14% (FIG. 10c). Since the spatial separation of the G bases at the 3′ end of AO-SS from TAMRA in the RO-TAMRA-CCO hybrid should be similar to that in the RO-TAMRA-CO hybrid, similar quenching effect of AO-SS on the emission from RO-TAMRA-CCO was observed (FIG. 10d). In the absence of a capture oligonucleotide, the addition of excess AO-SS to RO-TAMRA did not cause any observable change in the fluorescent emission of TAMRA. This confirms that there was no direct quenching of RO-TAMRA emission by AO-SS when they were separated in solution.

TABLE 5
Summary of the effect of AO-SS on the emission intensities
of the RO- TAMRA- CO and RO-TAMRA- CCO hybrids

| | Percent Decrease in |
|---|---|
| Solution | Emission Intensity (%) |
| RO-TAMRA -CO + AO-SS | ~14% |
| RO-TAMRA -CCO + AO-SS | ~12% |

### Example 6

### A Single Base Change in a 15-mer-Target Sequence can be Detected

The experiment described in this example was performed with a slightly modified technique. Here, 4 mL of a 1.0×10⁻⁸ M solution of RO-TAMRA in TE buffer was prepared. The solution was heated to 76° C. and then cooled to 18° C. using a temperature-controlled circulating water bath (Fisher Scientific model 9105). The temperature of the solution was monitored using a digital device (Omega Digicator model 410B-THC-C) equipped with a probe (Model LN2002 702A) that was inserted into the solution. Fluorescence emission spectra of the solution were recorded upon every two-degree decrease in temperature until a temperature of 18° C. was reached. Emission intensities were calculated with respective the emission of the RO-TAMRA solution at 18° C. The sequences of the nucleotides used in this example are provided in Table 6.

TABLE 6
Nucleotide sequences used in Example 6

| | |
|---|---|
| RO-TAMRA | 5′-TAMRA-linker-AAA ATA ACC ACC CAC CCA |
| | CCC |
| CO | GGG TGG GTG GGT GGT TAT TTT CCC TTA CAT |
| | CGT GGG TGC TTC CGT AAG GGT GGG AGG GAG |
| | GGA GGG AGA G |
| | (SEQ ID NO: 8) |
| B7-67mer | CCA GAA CTT ACG GAA GCA CCC ACG ATG GAC |
| | CCC AGA TGC ACC ATG GGC TTG GCA ATC CTT |
| | ATC TTT G |
| | (SEQ ID NO: 9) |
| T3 | GGA AGC ACC CAC GAT |
| | (SEQ ID NO: 10) |
| SM | GGA AGA ACC CAC GAT |
| | (SEQ ID NO: 11) |

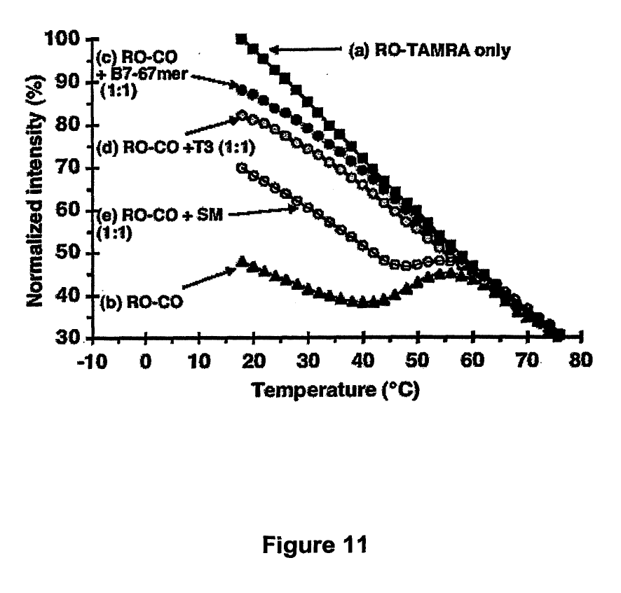

FIG. 11 shows that the emission intensity of RO-TAMRA increased with decreasing temperature because of reduced non-radiative decay. To demonstrate that a single base mismatch in a 15mer sequence complementary to a sequence in the loop of CO can be detected, a few μL of a 10⁻⁵ M solution of T3 or SM was added to a solution of the self assembled CO+RO-TAMRA (10⁻⁸ M) prepared as described above. The emission spectra of the solutions were monitored when the solutions were cooled from 76° C. The relative emission intensities of the solutions with respect to the maximum emission intensity of RO-TAMRA at 18° C. were calculated. The sequence of T3 is complementary to the CO loop region only and has a melting temperature of ˜60° C. SM differs from T3 in only one base at position 6. The presence of one mole equivalence of T3 trapped RO-TAMRA+CO in the hairpin opened form and increased the emission intensity to ˜80% at 18° C. Compared to T3, SM binds to TAMRA+CO at a lower temperature and is less effective in keeping RO-TAMRA+CO in the hairpin-opened form. Consequently, the emission profile of the RO-TAMRA+CO+SM hybrid differed significantly from that of RO-TAMRA+CO+T3 and less intense TAMRA emission was observed for RO-TAMRA+CO+SM at 18° C.

As summarized in Table 7, the emission intensity of RO-TAMRA+CO at 18° C. was 48% of the value obtained with RO-TAMRA alone (this value was normalized to 100%). CO+RO-TAMRA was prepared by adding a few μL of a concentrated solution (10⁻⁵ M) of CO to the solution of RO-TAMRA (10⁻⁸ M) to give one mole-equivalence of CO with respect to RO-TAMRA. The resultant solution was heated to 76° C. and then cooled to 18° C. Emission spectra of the solution were recorded upon every two-degree decrease in temperature until a temperature of 18° C. was reached. B7-67mer added before cooling maintained the stem-loop in the open configuration and gave an emission intensity of 88%. As expected, when T3 was added the smaller 15mer was slightly less effective at keeping the stem-loop in the open configuration (emission intensity of 82% at 18° C.). However, when SM was added, the emission intensity at 18° C. was only 70%, indicating that only a single base mismatch with the complementary sequence in CO can be detected. In a microarray application, this allows sequences differing in only a single base to be identified, and would forestall cross reactivities between similar nucleotide sequences, a major problem with current gene microarrays. It can also be used for the analysis of point mutations in gene sequences.

TABLE 7
A single base change in a 15mer target sequence can
be detected as a change in emission intensity

| | Normalized Emission Intensity |
|---|---|
| Solution | at 18° C. (%) |
| RO-TAMRA | 100 |
| RO-TAMRA + CO (1.0 equiv.) | 48 |
| RO-TAMRA + CO + B7-67mer | 88 |
| RO-TAMRA + CO + T3 | 82 |
| RO-TAMRA + CO + SM | 70 |

1. A nucleic acid array comprising a substrate and a nucleic acid complex, wherein said nucleic acid complex comprises:
a first nucleic acid molecule that is stably attached to the substrate; and
a second nucleic acid molecule that is hybridized to the first sequence, said second nucleic acid molecule comprising (1) a hairpin-forming sequence capable of forming a stem-loop and (2) a reporter-binding sequence capable of hybridizing under nucleic acid array hybridization conditions to a fluorophore-labeled reporter sequence,
wherein formation of said stem-loop modifies fluorescence signals of said fluorophore-labeled reporter sequence when said reporter sequence is hybridized to said second nucleic acid molecule.

2. The nucleic acid array of claim 1, wherein formation of said stem-loop quenches fluorescence signals of said fluorophore-labeled reporter sequence when said reporter sequence is hybridized to said second nucleic acid molecule.

3. The nucleic acid array of claim 2, wherein disruption of said stem-loop produces a detectable increase in fluorescence signals of said fluorophore-labeled reporter sequence when said reporter sequence is hybridized to said second nucleic acid molecule.

4. The nucleic acid array of claim 3, wherein said disruption is mediated by hybridization of a target sequence to said second nucleic acid molecule.

5. The nucleic acid array of claim 1, wherein said second nucleic acid molecule is hybridized to said fluorophore-labeled reporter sequence.

6. The nucleic acid array of claim 5, wherein said second nucleic acid molecule comprises said stem-loop.

7. The nucleic acid array of claim 5, wherein said second nucleic acid molecule does not comprise said stem-loop, and is hybridized to a target sequence.

8. The nucleic acid array of claim 1, wherein said second nucleic acid molecule comprises at least one guanine base, and formation of said stem-loop brings said at least one guanine base into close proximity to said fluorophore-labeled reporter sequence when said reporter sequence is hybridized to said second nucleic acid molecule, thereby quenching fluorescence signals of said reporter sequence.

9. The nucleic acid array of claim 8, wherein said at least one guanine base comprises two or more guanine bases.

10. The nucleic acid array of claim 8, wherein said second nucleic acid molecule comprises, from the 5′ end to the 3′ end, said reporter-binding sequence, said hairpin-forming sequence, said at least one guanine base, and a sequence that is hybridized to said first nucleic acid molecule.

11. The nucleic acid array of claim 8, wherein said second nucleic acid molecule comprises, from the 3′ end to the 5′ end, said reporter-binding sequence, said hairpin-forming sequence, said at least one guanine base, and a sequence that is hybridized to said first nucleic acid molecule.

12. The nucleic acid array of claim 1, wherein said first nucleic acid molecule is stably attached to different discrete regions on the nucleic acid array, wherein each said different discrete region comprises a different oligonucleotide that is hybridized to said first nucleic acid molecule, said oligonucleotide comprising (1) a hairpin-forming sequence capable of forming a stem-loop and (2) a reporter-binding sequence capable of hybridizing under nucleic acid array hybridization conditions to a fluorophore-labeled reporter sequence, wherein formation of said stem-loop quenches fluorescence signals of said fluorophore-labeled reporter sequence when said reporter sequence is hybridized to said oligonucleotide, and wherein each said different oligonucleotide is capable of hybridizing to a different target sequence.

13. The nucleic acid array of claim 12, wherein each said oligonucleotide comprises at least one guanine base, and formation of said stem-loop in said oligonucleotide brings said at least one guanine base into close proximity to said fluorophore-labeled reporter sequence when said reporter sequence is hybridized to said oligonucleotide, thereby quenching fluorescence signals of said reporter sequence.

14. The nucleic acid array of claim 12, wherein hybridization of one said target sequence to the corresponding oligonucleotide disrupts said stem-loop in the corresponding oligonucleotide, thereby producing a detectable increase in fluorescence signals of said fluorophore-labeled reporter sequence when said reporter sequence is hybridized to the corresponding oligonucleotide.

15. The nucleic acid array of claim 12, wherein said reporter-binding sequence in each said different oligonucleotide consists of the same nucleotide sequence.

16. A nucleic acid complex comprising an oligonucleotide hybridized to a fluorophore-labeled reporter sequence, wherein the oligonucleotide comprises a hairpin-forming sequence capable of forming a stem-loop, and wherein formation of the stem-loop modifies fluorescence signals of the reporter sequence when the reporter sequence is hybridized to the oligonucleotide.

17. The nucleic acid complex of claim 16, wherein formation of the stem-loop quenches fluorescence signals of the fluorophore-labeled reporter sequence when the reporter sequence is hybridized to the oligonucleotide.

18. The nucleic acid complex of claim 17, wherein disruption of the stem-loop produces a detectable increase in fluorescence signals of the fluorophore-labeled reporter sequence when the reporter sequence is hybridized to the oligonucleotide.

19. The nucleic acid complex of claim 18, wherein said disruption is mediated by hybridization of a target sequence to the oligonucleotide.

20. The nucleic acid complex of claim 19, wherein the oligonucleotide is hybridized to the target sequence and does not comprise the stem-loop.

21. The nucleic acid complex of claim 19, wherein the oligonucleotide comprises the stem-loop and is not hybridized to the target sequence.

22. The nucleic acid complex of claim 16, wherein the oligonucleotide comprises at least one guanine base, and formation of the stem-loop brings said at least one guanine base into close proximity to the fluorophore-labeled reporter sequence when the reporter sequence is hybridized to the oligonucleotide, thereby quenching fluorescence signals of the reporter sequence.

23. The nucleic acid complex of claim 22, wherein said at least one guanine base comprises two or more guanine bases.

24. The nucleic acid complex of claim 22, wherein the oligonucleotide comprises, from one end to the other end, a sequence that is hybridized to the fluorophore-labeled reporter sequence, the hairpin-forming sequence, and said at least one guanine base.

25. A method for detecting the presence or absence of a target sequence, comprising the steps of:
hybridizing an oligonucleotide to a nucleic acid sample and a fluorophore-labeled reporter sequence, wherein the oligonucleotide comprises (1) a hairpin-forming sequence capable of forming a stem-loop and (2) a sequence capable of hybridizing under nucleic acid array hybridization conditions to the fluorophore-labeled reporter sequence, wherein the oligonucleotide is capable of hybridization under nucleic acid array hybridization conditions to the target sequence, and hybridization of the oligonucleotide to the target sequence prevents formation of the stem-loop in the oligonucleotide, and wherein formation of the stem-loop quenches fluorescence signals of the fluorophore-labeled reporter sequence when the reporter sequence is hybridized to the oligonucleotide; and
detecting the fluorescent signals of the reporter sequence,
wherein an increase in fluorescence signals of the fluorophore-labeled reporter sequence in the presence of the nucleic acid sample compared to that in the absence of the nucleic acid sample is suggestive of the presence of the target sequence in the sample, and no significant change in fluorescence signals of the fluorophore-labeled reporter sequence in the presence of the nucleic acid sample compared to that in the absence of the nucleic acid sample is suggestive of the absence of the target sequence in the sample.

26. A method for detecting a sequence difference between a target sequence and a sequence of interest, comprising the steps of:
hybridizing an oligonucleotide to the sequence of interest and a fluorophore-labeled reporter sequence, wherein the oligonucleotide comprises (1) a hairpin-forming sequence capable of forming a stem-loop and (2) a sequence capable of hybridizing under nucleic acid array hybridization conditions to the fluorophore-labeled reporter sequence, wherein the oligonucleotide comprises a sequence that is complementary to the target sequence, and hybridization of the target sequence to the oligonucleotide prevents formation of the stem-loop in the oligonucleotide, and wherein formation of the stem-loop quenches fluorescence signals of the fluorophore-labeled reporter sequence when the reporter sequence is hybridized to the oligonucleotide; and
detecting the fluorescent signals of the reporter sequence,
wherein an decrease in fluorescence signals of the fluorophore-labeled reporter sequence in the presence of the sequence of interest compared to that in the presence of the target sequence, together with an increase in fluorescence signals of the fluorophore-labeled reporter sequence in the presence of the sequence of interest compared to that in the absence of the sequence of interest, is suggestive that the sequence of interest is homologous to but different from the target sequence.

27. The method of claim 26, wherein the target sequence differs from the sequence of interest by one single nucleotide mutation.

28. A nucleic acid array comprising a plurality of nucleic acid molecules, each of said nucleic acid molecules being stably attached to a different discrete region on the nucleic acid array, and each of said nucleic acid molecules comprising (1) a hairpin-forming sequence capable of forming a stem-loop and (2) a reporter-binding sequence capable of hybridizing under nucleic acid array hybridization conditions to a fluorophore-labeled reporter sequence, wherein formation of said stem-loop in one said nucleic acid molecule modifies fluorescence signals of said fluorophore-labeled reporter sequence when said reporter sequence is hybridized to said one nucleic acid molecule.

29. The nucleic acid array of claim 28, wherein formation of said stem-loop in said one nucleic acid molecule quenches fluorescence signals of said fluorophore-labeled reporter sequence when said reporter sequence is hybridized to said one nucleic acid molecule.

30. The nucleic acid array of claim 29, wherein disruption of said stem-loop in said one nucleic acid molecule produces a detectable increase in fluorescence signals of said fluorophore-labeled reporter sequence when said reporter sequence is hybridized to said one nucleic acid molecule.

31. The nucleic acid array of claim 30, wherein said disruption is mediated by hybridization of a target sequence to said one nucleic acid molecule.

32. The nucleic acid array of claim 28, wherein said one nucleic acid molecule is hybridized to said fluorophore-labeled reporter sequence.

33. The nucleic acid array of claim 32, wherein said one nucleic acid molecule comprises said stem-loop.

34. The nucleic acid array of claim 32, wherein said one nucleic acid molecule does not comprise said stem-loop, and is hybridized to a target sequence.

35. The nucleic acid array of claim 28, wherein said one nucleic acid molecule comprises at least one guanine base, and formation of said stem-loop brings said at least one guanine base into close proximity to said fluorophore-labeled reporter sequence when said reporter sequence is hybridized to said one nucleic acid molecule, thereby quenching fluorescence signals of said reporter sequence.

36. The nucleic acid array of claim 35, wherein said at least one guanine base comprises two or more guanine bases.

37. The nucleic acid array of claim 35, wherein said one nucleic acid molecule comprises, from the 5′ end to the 3′ end, said reporter-binding sequence, said hairpin-forming sequence, and said at least one guanine base.

38. The nucleic acid array of claim 35, wherein said one nucleic acid molecule comprises, from the 3′ end to the 5′ end, said reporter-binding sequence, said hairpin-forming sequence, and said at least one guanine base.

39. nucleic acid array of claim 28, wherein each of said nucleic acid molecules is covalently attached to the different discrete region on the nucleic acid array.

40. nucleic acid array of claim 28, wherein each of said nucleic acid molecules is non-covalently attached to the different discrete region on the nucleic acid array.
